(19) United States
(12) Plant Patent Application Publication
(10) Pub. No.: US 2012/0304345 P1
(43) Pub. Date: Nov. 29, 2012

(54) Medinilla hybrid named 'Bella'

(50) ;

(75) Inventors: Elly Bak, Rijsenhout (NL); Nicolaas D.M. Steur, Oude Niedorp (NL)

(73) Assignee: CORN.BAK B.V.

(21) Appl. No.: 13/067,309

(22) Filed: May 24, 2011

(51) Int. Cl. A01H 5/00

(52) U.S. Cl. PLT/226

(57) ABSTRACT

A new and distinct Medinilla sp. plant named ‘BELLA’, characterized by an erect shrub plant, measuring about 50 cm in total height (when first flowering) and about 55 cm in diameter; green colored leaves, measuring about 20 cm in length and about 8 cm in width; pendulous panicles, measuring about 20 cm in length and about 15 cm in diameter, with pink (RHS 78D) flowers.

### LATIN NAME OF THE GENUS AND SPECIES OF THE PLANT CLAIMED

Medinilla sp.

### VARIETY DENOMINATION

‘BELLA’

### BACKGROUND OF THE INVENTION

The present invention relates to a new and distinct hybrid cultivar of Medinilla plant, botanically known as Medinilla sp., hereinafter referred to by the cultivar name ‘BELLA’. The genus of Medinilla belongs to the Melastomataceae family.

The new Medinilla ‘BELLA’ is a product of a planned breeding program conducted by the inventors, Elly Bak and Nico D. M. Steur, in Assendelft, The Netherlands. The objective of the breeding program is to develop a new Medinilla variety with good growing habit and attractive inflorescence.

The new Medinilla ‘BELLA’ originated from a cross made in a controlled breeding program by the inventors in 2007 in Assendelft, The Netherlands. The female or seed parent is the Medinilla selection designated ‘82954514’ (unpatented), and the male or pollen parent is the Medinilla selection designated ‘82957015’ (unpatented). ‘BELLA’ was discovered and selected by the inventors in 2008, as a single flowering plant within the progeny of the stated cross in a controlled environment in Assendelft, The Netherlands.

Asexual reproduction of the new Medinilla hybrid ‘BELLA’ performed by vegetative means by cuttings was first performed in 2008, in Assendelft, The Netherlands. The first ‘BELLA’ plants propagated through the use of cuttings flowered in 2009, in Assendelft, The Netherlands, and have demonstrated that the combination of characteristics as herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be unique characteristics of ‘BELLA’ which in combination distinguish this Medinilla hybrid as a new and distinct cultivar:

1. Solid growth habit;

2. Erect shrub plant, measuring about 50 cm in height (above the pot when first flowering);

3. Green colored leaves, measuring about 20 cm in length and about 8 cm in width;

4. Pendulous panicles with pink (RHS 78D) colored flowers;

Of the many commercial cultivars known to the present inventors, the most similar in comparison to the new Medinilla ‘BELLA’ is the Medinilla magnifica PINATUBO′ (unpatented). Plants of the new Medinilla ‘BELLA’ differ from plants of Medinilla ‘PINATUBO’ primarily in leaf size, bract size, and inflorescence color. Plants of ‘BELLA’ have smaller leaves and bracts than plants of ‘PINATUBO’. Additionally, plants of ‘BELLA’ have darker pink inflorescence (RHS 75B) than plants of ‘PINATUBO’ (RHS 72C).

### BRIEF DESCRIPTION OF THE PHOTOGRAPHS

The patent or application file contains at least one drawing executed in color. Copies of this patent or patent application publication with color drawing(s) will be provided by the Office upon request and payment of the necessary fee.

The accompanying photograph illustrates the overall appearance of the new Medinilla ‘BELLA’ showing the colors as true as is reasonably possible with colored reproductions of this type. Colors in the photograph may differ slightly from the color values cited in the detailed botanical description which accurately describes the color of ‘BELLA’.

FIG. 1 shows a side view perspective of a typical potted flowering plant of ‘BELLA’ at about 50 weeks from potting.

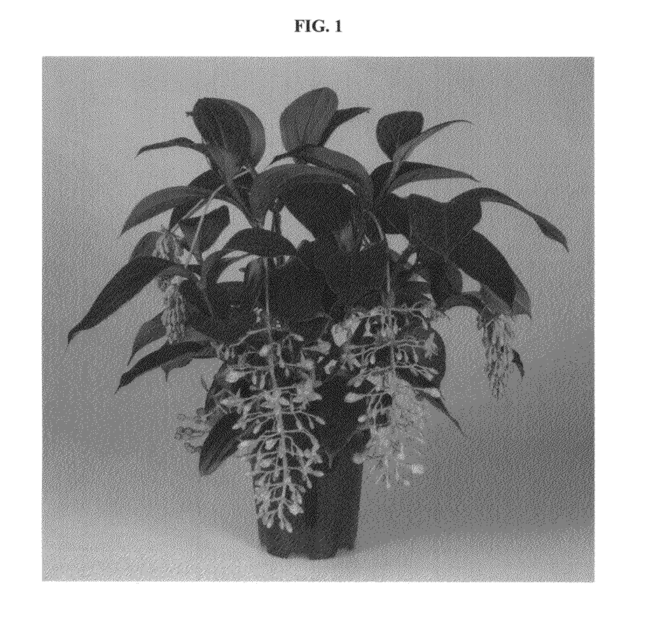

### DETAILED BOTANICAL DESCRIPTION

‘BELLA’ has not been observed under all possible environmental conditions. The phenotype of the new cultivar may vary with variations in environment such as temperature, light intensity, day length, fertilizers and humidity without any change in the genotype of the plant.

The aforementioned photograph, together with the following observations, measurements and values describe the new Medinilla ‘BELLA’ as grown in a greenhouse in Assendelft, The Netherlands, under conditions which closely approximate those generally used in commercial practice.

Color references are made to The Royal Horticultural Society Colour Chart (R.H.S.), 2001 Edition, except where general colors of ordinary significance are used. Color values were taken under daylight conditions in a greenhouse in Assendelft, The Netherlands. The age of the plants of ‘BELLA’ described is about 50 weeks from potting.
Classification:
   Botanical.—Medinilla sp.
Parentage:
   Female parent.—Medinilla selection designated ‘82954514’ (unpatented).
   Male parent.—Medinilla selection designated ‘82957015’ (unpatented).
Propagation: Cuttings
Plant:
   General appearance and form.—Height: About 50 cm Diameter: About 55 cm Shape: Erect shrub.
   Growth habit.—stem.
   Plant vigor.—good.
   Flowering season.—from April until October.
   Cold tolerance.—frost tender, temperatures below 5° C. may damage plants.
   Fragrance.—none.
Foliage:
   Quantity.—About 30-35.
   Size.—Length: About 15-20 cm. Width: About 8 cm.
   Overall shape.—elliptical.
   Apex shape.—cuspidate.
   Base shape.—acute.
   Margin.—entire.
   Texture.—smooth.
   Color.—Leaf color can vary somewhat depending on growing conditions. Upper Surface: Glossy green, RHS 137A-139A Lower Surface: Green, RHS 137C-143A.
   Venation.—countersunk.
Inflorescence:
   Shape.—Pendulous panicles.
   Amount.—about 2-6 (depending on the size of the plant).
   Size.—Length: About 20 cm Width: About 15 cm.
   Color.—Pink, closest to RHS 78D.
   Corolla.—Number: about 120 per panicle.
   Petals.—Number: 4 per flower Length: 12 mm Width: 8 mm Overall Shape: obovate, asymmetrical Apex Shape: obtuse Color: Upper surface: pink, closest to RHS 78D Lower surface: pink, closest to RHS 78D Sepals: growth together, enclosing the ovary. Color: red purple, closest to RHS 57C.
   Bracts.—Quantity: 4 per crown Arrangement: verticillate Size: Length: about 22 mm Width: about 13 mm Overall shape: elliptical.
Reproductive organs:
Androecium:
   Stamen.—Number: 10 per flower Length: 6 mm Diameter: 1 mm Color: light yellow, closest to RHS 11 C.
   Anther.—Length: 10 mm Color: violet, closest to RHS 87B, with a yellow base.
   Pollen.—Amount: not visible.
   Gynoecium.—
   Pistil.—Number: 1 per flower Length: 20 mm.
   Stigma.13 Width: <1 mm Color: white, too small to qualify RHS value.
   Style.13 Length: 17 mm Color: white, closest to RHS 155C.
   Ovary.13 Shape: round Position: inferior Length: 8 mm Diameter: 6 mm Color: white, closest to RHS 155C.
Seeds: No seeds produced
Fruit: No fruit produced
Disease/pest resistance: No observations made
Disease/pest susceptibility: No observations made

What is claimed is:

1. A new and distinct Medinilla plant named ‘BELLA’, as illustrated and described herein.
